(19) United States
(12) Patent Application Publication
(10) Pub. No.: US 2025/0054149 A1
(43) Pub. Date: Feb. 13, 2025

(54) METHOD OF MEASURING PALPEBRAL FISSURE HEIGHT, AND A DEVICE AND A STORAGE MEDIUM FOR THE SAME

(71) Applicants: SHANGHAI INSTITUTE OF ENDOCRINE AND METABOLIC DISEASES, Shanghai (CN); SHANGHAI BAIYI HEALTHCARE TECHNOLOGY CO., LTD ., Shanghai (CN)

(72) Inventors: Weiqing WANG, Shanghai (CN); Guang NING, Shanghai (CN); Liyun SHEN, Shanghai (CN); Chaonan TIAN, Shanghai (CN); Dong DU, Shanghai (CN)

(21) Appl. No.: 18/933,132

(22) Filed: Oct. 31, 2024

Related U.S. Application Data
(63) Continuation of application No. PCT/CN2023/113527, filed on Aug. 17, 2023.

(30) Foreign Application Priority Data
Aug. 18, 2022 (CN) 202210989506.4

(51) Int. Cl. G06T 7/00; A61B 3/14; G06T 7/11

(52) U.S. Cl. CPC G06T 7/0012; A61B 3/14; G06T 7/11; G06T 2207/10048; G06T 2207/20081; G06T 2207/20084; G06T 2207/30041

(57) ABSTRACT

The application relates to a method, device and storage medium for measuring palpebral fissure height. The method comprises capturing the first eye position image of the user looking straight ahead in a near-infrared light field of 700-1200 nm at the position in front of the eye; and segmenting the background, the iris, the sclera and the pupil from the first eye position image by the training method of a neural network. The pupil center was extracted from the segmented pupil, and the vertical pupil center line was obtained. The distance between the junction point of the sclera, the iris or the pupil on the center line of the pupil and the background is obtained, and the palpebral fissure height is calculated using the distance.

### CROSS-REFERENCE TO RELATED APPLICATIONS

This application is a continuation of International Pat. Appl. No. PCT/CN2023/113527, filed on Aug. 17, 2023, which claims priority to Chinese Pat. Appl. No. 202210989506.4, filed on Aug. 18, 2022, the contents of each of which are incorporated by reference herein in their entireties.

### TECHNICAL FIELD

The invention relates to the technical field of eye measurement, in particular to a method of measuring palpebral fissure height, and a device and a storage medium for the same.

### BACKGROUND DISCUSSION

Palpebral fissure vertical height (PFH, or eye or eyelid crack width) refers to the distance between the upper and lower eyelid edges, passing through the pupil. Clinically, eyelid retraction, including upper and lower eyelid retraction, is a common clinical symptom of thyroid-associated ophthalmopathy (TAO) and an important indicator in clinical diagnoses. Eyelid retraction may not only cause unacceptable facial damage, but also lead to vision-threatening exposed keratopathy, such as corneal ulcers. Therefore, the accurate measurement and detection of eyelid retraction is very important for clinical diagnoses, and the degree of eyelid retraction can be reflected by measuring the palpebral fissure height.

At present, palpebral fissure height is usually measured on a millimeter scale in the clinic, but errors in the measurement data are easy to make due to inaccurate readings and the influence of the operator's experience level and practices. However, in the diagnosis and evaluation of TAO patients, a fluctuation of 2 mm often predicts a change in the patient's condition, so measurement accuracy is often required, and the manual measurement methods can cause missed diagnoses and misdiagnosis of the patient, delaying progress in the treatment of the patient's condition.

In addition, the prior art also uses automatic measurement to determine the occurrence of eyelid retraction. For example, in a Chinese patent entitled “Method and Device for Identification of Ocular Signs in Thyroid Related Eye Diseases” (Application No. 202010803761.6, publication date: Oct. 30, 2020), the cornea and the sclera are identified through image recognition and neural network training, and then exposure of the upper eyelid and the upper margin of the cornea region to the sclera region is determined from the image of the cornea and the sclera, to determine whether there is an eyelid retraction. However, this method can cause misjudgments for people with Sanpaku eyes, or those who show the whites of the eyes due to the protrusion of the eyeball caused by myopia. For example, in a Chinese patent entitled “Blink frequency analysis method and system based on image processing” (Application No. 201910939612.X, publication date: Feb. 4, 2020), the iris contour and the sclera contour are determined by taking human eye images, the eyelid margins (edges) are determined by the iris contour and the sclera contour, and the PFH is calculated by the difference between the coordinates of the uppermost point of the upper eyelid margin and the coordinates of the lower node. However, the PFH is defined as the distance between the upper and lower eyelid edges through the center line of the pupil, and the PFH determined in the above way may include an oblique distance between eyelid margins.

Although there are prior arts that use neural network model training methods to segment eye images, their training models are relatively old, and the training process is time-consuming and occupies large amounts of memory space. Therefore, it is necessary to further improve the technologies.

### SUMMARY

In order to overcome the lack of accuracy of measuring the palpebral fissure vertical height (hereinafter, “palpebral fissure height” or PFH) in the prior art, a method for measuring the palpebral fissure height is disclosed in this application.

In order to achieve the above purposes, the following technical solutions are adopted in this application:

On one hand, a method for measuring a palpebral fissure height is disclosed, which comprises:

Acquiring a first eye position image of a user in front of an eye (e.g., of the user), looking straight ahead in a near-infrared light field of 700-1200 nm;

Segmenting a background, an iris, a sclera and a pupil from the first eye position image by a training method of a neural network;

Extracting a center of the pupil from the segmented pupil and obtaining a center line of the pupil in a vertical direction; and

Determining a distance between a junction point of the sclera, the iris or the pupil on the center line of the pupil and the background, and calculating the palpebral fissure height using the distance.

Further, the pupil center may be an average value of X and Y coordinates of all pixels of the pupil segmented from the first eye position image, and the center line of the pupil may be obtained by drawing a vertical line through an average value of the X coordinates.

Further, the training method of the neural network may segment the background, the iris, the sclera and the pupil from the first eye position image by adopting a combination of UNet and DenseNet neural network models as the neural network model, taking the first eye position image as an input to the neural network model, adopting the UNet neural network model to reduce and increase a dimension of the input, transmitting an output of a dimensionality reducing block to a corresponding dimensionality increasing block by a skip connection, and extracting each dimensionality reducing block and upsampling each dimensionality increasing block using the DenseNet neural network model.

Further, the method may also include verifying a training effect of the neural network using a loss function, wherein the loss function is a compound loss that comprises a focal loss, a generalized dice loss, a surface loss and a boundary aware loss.

The loss function may be calculated as follows:

$$Compound loss = {a}_{1} \times surface loss + {a}_{2} \times focal loss + {a}_{3} \times generalized dice loss + {a}_{4} \times boundary aware loss \times focal loss$$

wherein α₁, α₂, α₃ and α₄ are hyperparameters, α₁ is related to a number of duration(s) during training, α₂=1, α₃=1−α₁, and α₄=20.

Further, the palpebral fissure height may be calculated as: PFH (B=a pixel distance (A) of the palpebral fissure height×a single pixel width or length×a distance (D) from the palpebral fissure to a lens of the camera÷a distance (C) from a film in the camera to the lens of the camera, where the pixel distance (A) of the palpebral fissure height is the distance between the junction point of the sclera, the iris, or the pupil on the center line of the pupil and the background.

Specifically, the distance (D) from the palpebral fissure to the lens of the camera may equal the distance from a corner of the eye to the lens of the camera (1), minus an ocular prominence or exophthalmia (2).

Further, the ocular prominence or exophthalmia (2) may be an average value of a normal eye (e.g., the average ocular prominence of a normal eye).

On the one hand, the present application also provides a device for measuring palpebral fissure height, including:

An image acquisition module, configured to acquire a first eye position image of a user looking straight ahead in a near-infrared light field of 700-1200 nm in front of an eye;

An image segmentation module, configured to adopt a training mode of a neural network to segment the background, the iris, the sclera and the pupil from the first eye position image;

A feature extraction module, configured to extract a pupil center from the segmented pupil and obtain a pupil center line in the vertical direction; and

A computing module, configured to calculate the distance between the junction point of the sclera, the iris or the pupil and the background on the pupil center line, and calculate the palpebral fissure height from the distance.

On another hand, the present application discloses a computer-readable storage medium, configured to have at least one program code that is loaded and executed by a processor to perform the present method of measuring palpebral fissure height.

Compared with the prior art, the proposed technical solution has at least the following beneficial effects:

1. Taking eye images in the near-infrared light field can effectively distinguish the iris, the pupil, the sclera, the background and the lacrimal caruncle, so as to provide more intuitive and more recognizable images for subsequent neural network training.

2. This application adopts a combination form of UNet and DenseNet neural network models for neural network training, which not only takes advantage of the simple and stable structure of the UNet neural network model, which is widely used in medical image processing, but also applies the DenseNet neural network model in each dimensionality reducing block and dimensionality increasing block of the UNet neural network model. In each dimensionality reducing block and dimensionality increasing block, feature reuse is carried out through a skip connection to improve the feature extraction effect. Moreover, the DenseNet neural network model adopted in this application simplifies the existing DenseNet neural network model, avoiding the problem of slow calculation caused by the traditional DenseNet neural network model, which may be associated with all of the previous layers in each layer.

3. The iris, the pupil, the sclera, the background and the lacrimal caruncle can be accurately identified through the neural network model, so as to identify the intersection (or junction point) of the sclera, the iris or the pupil on the center line of the pupil and the background. The distance between the two junction points is the pixel distance of the palpebral fissure height, and the distance of the palpebral fissure height can be obtained through one or more geometric calculations.

### BRIEF DESCRIPTION OF THE DRAWINGS

FIG. 1 shows a flow chart of an exemplary method of measuring palpebral fissure height;

FIG. 2 shows a structure of an exemplary neural network model;

FIG. 3 shows an exemplary processing flow diagram of a dimensionality reducing block;

FIG. 4 shows an exemplary processing flow diagram of a dimensionality increasing block;

FIG. 5 shows a diagram of the principle(s) for calculating the palpebral fissure height; and

FIG. 6 is a schematic diagram of an exemplary device for measuring palpebral fissure height.

### EXAMPLES

Combined with the attached drawings and examples, the following specific implementation of this application is further described in detail. The following embodiments are used to illustrate, but not to limit, the scope of the application.

Based on neural network model training, this application extracts the features of the eye image taken to find out the pupil center and further obtain the pupil center line. The pixel distance of the palpebral fissure height may be determined according to the junction point of the pupil center line and the eye socket, and the palpebral fissure height is further calculated through one or more imaging principles. The concept(s) of this application is further explained by specific embodiments below.

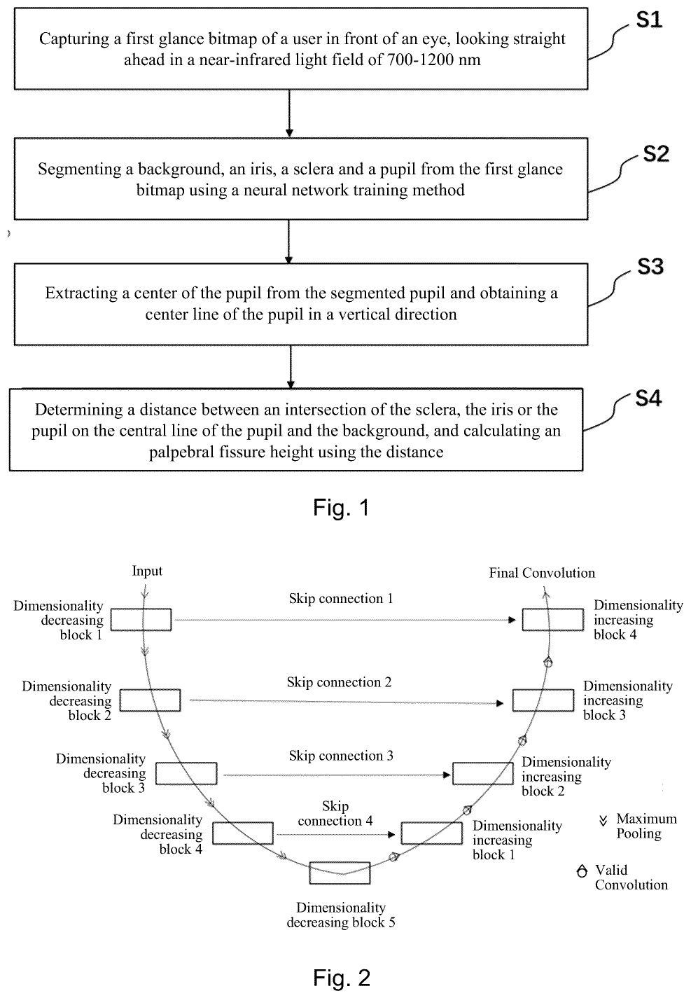

On the one hand, the method of measuring palpebral fissure height as shown in FIG. 1 includes the following steps:

Step S1: Acquiring the first eye position image of a user looking straight ahead in a near-infrared light field of 700-1200 nm.

In the above Step S1, the eye image is taken in the near-infrared light field of 700-1200 nm. In the normal visible light band of 400-700 nm, the color of different parts of the eye, namely the pupil, the iris, and the sclera, has almost no effect on the imaging of the eye, due to the gradual structure of the limbus of the cornea in the contact part of the iris and the sclera, resulting in difficulties or an inability to accurately identify the center of the eyeball. An absorption peak of human melanin pigment occurs at about 335 nm, and wavelengths over 700 nm are almost completely unabsorbed, but the reflectivity of the iris is quite stable in near-infrared wavelengths over 700 nm. Therefore, the use of near-infrared light field can easily distinguish boundaries between the sclera, the iris and the pupil. Thus, the accuracy and stability of the method can be improved.

Step S2: Using the training method of neural network to segment or distinguish the background, the iris, the sclera and the pupil from the first eye position image.

In the above Step S2, the neural network model adopts a combination of UNet and DenseNet neural network models. The neural network model takes the first eye position image as an input, adopts the UNet neural network model to reduce and increase the dimension(s) or dimensionality of the input, and transmits the output of the dimensionality-reducing block(s) to the corresponding dimension increasing block(s) by a skip connection. The DenseNet neural network model is used for feature extraction in each dimensionality reducing block, and for upsampling each dimensionality increasing block.

The neural network architecture adopted in this application is a combination of UNet and DenseNet neural network models, which is implemented based on a Pytorch framework. The overall structure of the neural network is a U-shape, as shown in FIG. 2, with a total of five dimensionality reducing blocks, from dimensionality reducing block 1 to dimensionality reducing block 5, and four dimensionality increasing blocks, from dimensionality increasing block 1 to dimensionality increasing block 4.

The neural network receives an input image and passes it to the dimensionality reducing blocks 1 through 5. With the exception of the dimensionality reducing block 1, each dimensionality reducing block is connected by a maximum pooling layer that reduces the dimensionality (e.g., number of dimensions of the image data) by half. The dimensionality reducing block 5 then passes the output to the dimensionality increasing blocks 1 through 4. The dimensionality increasing blocks 1 through 4 may be connected by a valid convolution or valid convolution layer that increase the dimensionality (e.g., number of dimensions of the image data) by two. The dimensionality increasing block 4 passes the output to a final convolution. Skip connections are listed as follows: the dimensionality reducing block 1 is connected to the dimensionality increasing block 4; the dimensionality reducing block 2 is connected to the dimensionality increasing block 3; the dimensionality reducing block 3 is connected to the dimensionality increasing block 2; and the dimensionality reducing block 4 is connected to the dimensionality increasing block 1.

UNet provides a deep convolutional structure, firstly reducing the input dimension (e.g., dimensions of input data) to or by a certain degree, and then bumping the input back to the original size or number of dimensions. At the same time, UNet can transmit information from the dimensionality reducing blocks to corresponding dimensionality increasing blocks by skip connections, and the output of the early layer(s) may be passed to the later layer(s) as an input, which can prevent information loss inside the neural network, and allow maximum learning. On the other hand, DenseNet is more of a concept than a fixed structure. The focus of DenseNet in this application is the specific skip connection technology. An optimized and scaled-down version may be applied in this application. Instead of taking the output of all previous layers as input, as is the case for each layer of the traditional DenseNet network model, each dimensionality increasing and reducing block may perform the skip connection operation 1 or 2 times, which prevents the network from being too slow.

In order to explain the neural network training process, the following concepts are introduced:

Max Pooling: Max pooling is a pooling operation that calculates the maximum value in each patch of each feature map. The result is a downsampled or pooled feature map that highlights the most features in the patch, rather than the average presence of the features in the average pooled case. This operation corresponds to the torch.nn.MaxPool2d function. The kernel size is defined as 2×2, and this operation halves the input dimensionality.

Convolution: Convolution is the simple application of a filter (also known as a kernel) to an input, resulting in an activation. Repeated application of the same filter to the input produces an activation graph, called a feature graph, indicating the location and intensity of the detected features in the input. This operation corresponds to the torch.nn.Conv2d function.

Same Convolution: The same convolution is a type of convolution where the output matrix has the same dimensionality as the input matrix. This operation corresponds to the torch.nn.Conv2d function, which, in the example of this application, has a padding of 1×1 and a kernel size of 3×3.

Valid Convolution: A valid convolution is a convolution operation that does not use any padding on the input. This operation corresponds to the torch.nn.Conv2d function, which has no padding in the example of this application and has a kernel size of 1×1.

Transposed Convolution: The transposed convolution layer attempts to reconstruct the spatial dimension of the convolution layer and inverts the downsampling and upsampling techniques applied to it. This operation corresponds to torch.nn.ConvTranspose2d.

Batch normalization: Batch normalization is a way to make artificial neural networks faster and more stable by re-centering and re-scaling the inputs of various layers. This operation corresponds to BatchNorm2d with num_features of 5, which is the number of labels or layers for the network.

Leaky RELU: The leaky correction linear activation function, or simply Leaky RELU, is a linear function that will output an input directly if the input is positive; otherwise, it will output the input, multiplied by a small factor (0.1 in the example of this application).

The working process of the exemplary dimensionality increasing blocks and dimensionality reducing blocks is as follows.

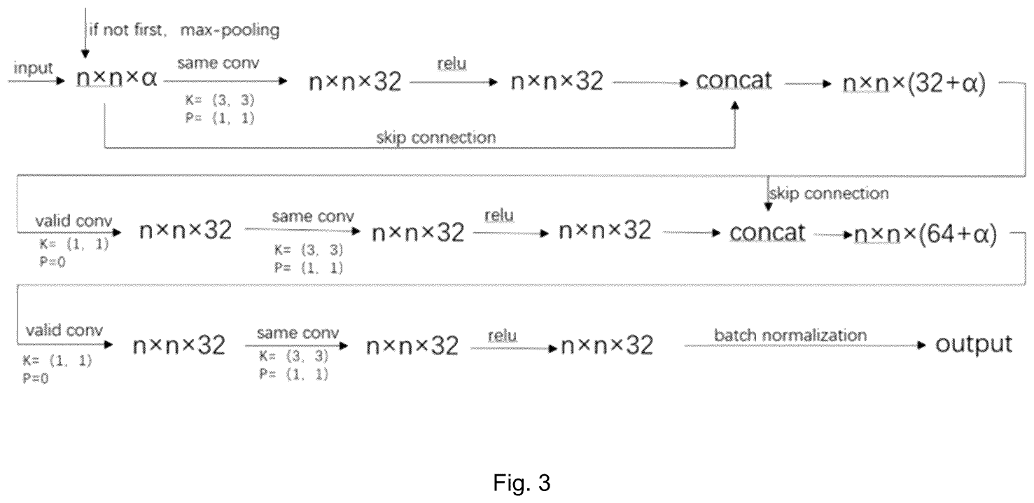

One or more (e.g., several) convolution operations, as well as Leaky RELU and skip connection operations, are applied to the dimensionality reducing blocks, or by the dimensionality reducing blocks to the input data. This application takes the size of the original image as n×n×1 dimensions as an example, in which the dimension 1 means that the original image has only gray values. A specific structure for the exemplary dimensionality reducing blocks is shown in FIG. 3.

First, the original input to the exemplary dimensionality reducing block passes through a same convolutional layer (Layer-1) with 32 filters (channels). The output of Layer-1 is then subjected to Leaky RELU activation (RELU-Layer-1). Second, RELU-Layer-1 is concatenated with the original input along the channel dimension(s) (Skip-Connection-Input-1), which makes the number of output dimensions 32 plus the number of dimensions of the original input, which is the first case where the skip connection is applied: the original input is passed directly to the later convolution stage.

Third, Skip-Connection-Input-1 is passed to a valid convolution layer (Layer-2) with 32 filters, reducing the dimensionality to 32, which is often referred to as the “bottleneck layer.” Layer-2 shrinks the channel dimensionality of the neural network to ensure that the network does not become too slow. Fourth, the output of Layer-2 passes again through a same convolutional layer (Layer-3) with 32 filters (channels). The output of Layer-3 is then subjected to a leaky RELU activation (RELU-Layer-3).

Fifth, RELU-Layer-3 is connected to RELU-Layer-1 and the original input (Skip-Connection-Input-2), which is the case when the skip connection is applied for the second time, and which concatenates the connected layers/data to increase the dimensionality to 64 plus the number of dimensions of the original input. Sixth, Skip-Connection-Input-2 again passes through a valid convolution layer (layer-4) with 32 filters, which again reduces the dimension to 32 dimensions. Seventh, the output of Layer-4 is again passed through the same convolutional layer (Layer-5) with 32 filters (channels). A leaky RELU activation (RELU-Layer-5) is then applied to the output of Layer-5. Finally, the result of RELU-Layer-5 may be passed to or through a batch normalization layer and output from the dimensionality reducing block. Subsequent dimensionality reducing blocks may further reduce the dimensionality of the corresponding output by half.

Also, before any convolution is applied, each dimensionality reducing block except for Dimensionality reducing block 1 (FIG. 2) may apply a maximum pooling layer to the input, since the input to Dimensionality reducing block 1 is the original image itself. This is also where “dimensionality reducing” may occur, as this layer can reduce the dimension of the input by half. The dimensionality increasing blocks restore the dimensionality (e.g., of the image data) to its original dimensionality.

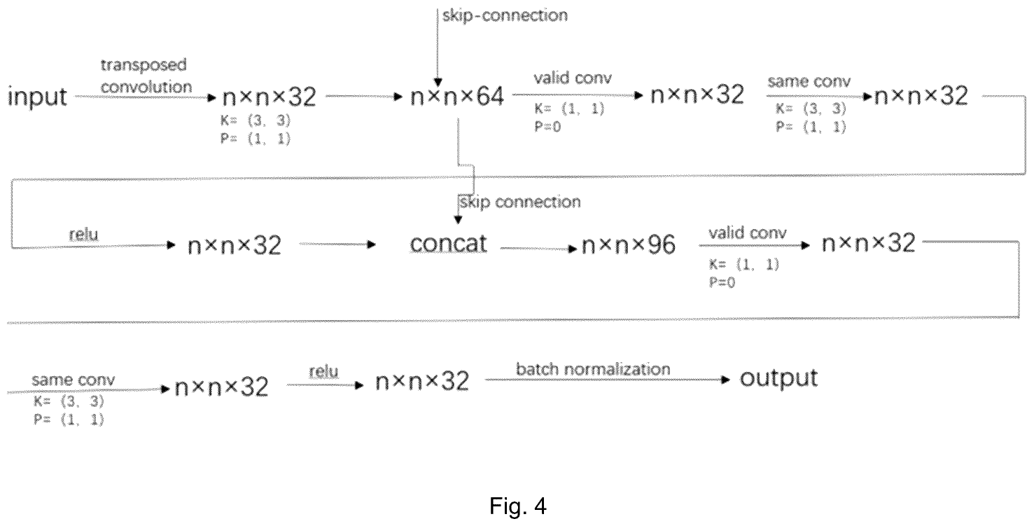

The dimensionality increasing blocks are similar to the dimensionality reducing blocks, except that they apply one or more valid (e.g., transposition) convolutions instead of maximum pooling, and the dimensionality increasing blocks may also receive a skip connection from one of the dimensionality reducing blocks (e.g., a corresponding dimensionality reducing block). A specific structure for the exemplary dimensionality increasing blocks is described below, and also shown in FIG. 4.

First, the exemplary dimensionality increasing block applies a transposition convolution (Layer-1) to the input, doubling the dimension of the input; This is where dimensionality increasing may occur. Second, the output of Layer-1 is connected along the channel dimension (Skip-Connection-Input-1) with the skip connection from the corresponding dimensionality reducing block (see FIG. 2). This is an example of a structural skip connection.

Third, referring back to FIG. 4, a valid convolution (Layer-2) with 32 filters reduces the dimensions Skip-Connection-Input-1 to 32 dimensions. Fourth, the output of Layer-2 passes again through a same convolutional layer (Layer-3) with 32 filters (channels). A leaking RELU activation (RELU-Layer-3) is then applied to the output of Layer-3.

Fifth, RELU-Layer-3 is skip-connected with Skip-Connection-Input-1, which increases the number of output dimensions to 96 (Skip-Connection-Input-2). Sixth, Skip-Connection-Input-2 again passes through a valid convolution layer (Layer-4) with 32 filters, again reducing the number of dimensions to 32. Seventh, the output of Layer-4 is again passed through a same convolutional layer (Layer-5) with 32 filters (channels). A leaking RELU activation (RELU-Layer-5) is then applied to the output of Layer-5. Finally, the result of RELU-Layer-5 is passed to or through a batch normalization layer and output from the dimensionality reducing block. Subsequent dimensionality increasing blocks may further increase the dimensionality of the corresponding output by two.

After the dimensionality increasing blocks and the dimensionality reducing blocks, the output will be an N×N×32-dimensional matrix, where N and N are the dimensions of the image. The N×N×32-dimensional matrix will pass through the final valid convolution of 5 filters (channels), and the final output is an N×N×5 matrix. Intuitively, this matrix assigns each pixel one of five values that represent the probability that the pixel should have a particular label. For example, 0 may be the value of the label for the background, 1 may be the value of the label for the sclera, 2 may be the value of the label for the iris, 3 may be the value of the label for the pupil, and 4 may be the value of the label for the lacrimal caruncle. The final output is calculated by finding the maximum of the 5 values for each pixel and assigning the index of these maximum values to an N×N matrix, which is the final output mask.

Step S3: Extract the pupil center from the segmented pupil and obtain the pupil center line in the vertical direction.

In the above Step S3, the pupil center is the average X and Y coordinates of all pupil pixels segmented or identified from the first eye position image, and the pupil center line can be obtained by determining the average of the X coordinates and drawing a vertical line through the average of the X coordinates. Alternatively, the pupil center line can be obtained by determining the average of the X coordinates for a plurality of Y coordinates, then drawing a vertical line that best fits the average of the X coordinates for each of the Y coordinates.

Step S4: Find the distance between the junction point(s) of the sclera, the iris or the pupil on the center line of the pupil and the background, and use the distance to calculate the palpebral fissure height. For example, one may first identify the locations on the image where (1) the sclera, the iris or the pupil intersects the center line of the pupil and (2) the background intersects the center line of the pupil, then determine or calculate the distance between the two locations on the image. In this example, the innermost border of the background may be the eyelid margin or edge in the image, and the location on the image where the sclera, the iris or the pupil intersects the center line of the pupil may be the location giving the largest value of A and/or B, or the location on the image where the sclera, the iris or the pupil intersects both (i) the center line of the pupil and (ii) the background or the eyelid margin or edge.

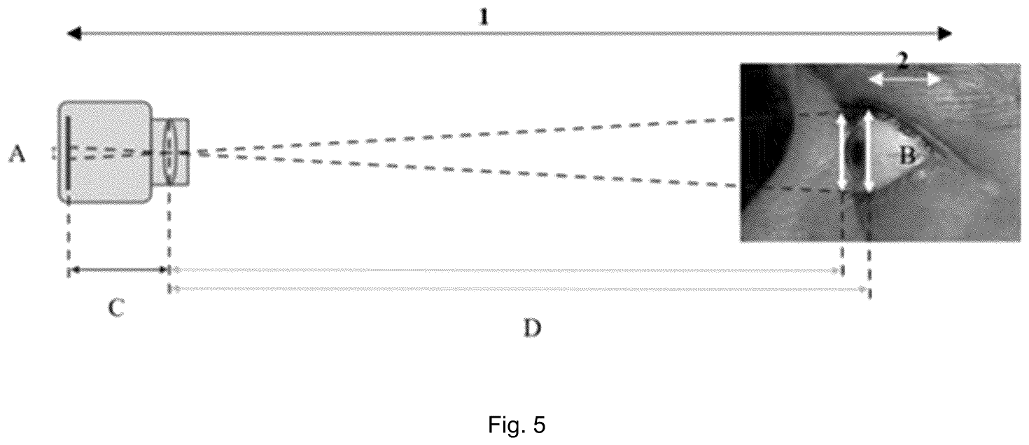

As shown in FIG. 5, the PFH is calculated according to the principle of similar triangles in Step S4, and the calculation formula is as follows:

Palpebral fissure height B=the pixel distance A of the palpebral fissure height×the width or length of a single pixel×the distance D from the palpebral fissure to the camera lens÷the distance C from the camera lens to the imaging device in the camera (e.g., an image sensor or other photodetector), wherein the pixel distance A of the palpebral fissure height is the distance between the junction point of the sclera, the iris or the pupil on the center line of the pupil and the background.

Further, in Step S4, the distance D from the palpebral fissure to the camera lens=the distance 1 from the corner (e.g., the outer canthus) of the eye to the camera lens-the ocular prominence or exophthalmia 2.

Further, the eyeball protrusion or exophthalmia 2 may be the average value of the exophthalmia 2 of normal (e.g., non-diseased) eyes. From the point of view of statistical averages, the average of normal ocular prominence is 12-14 mm. In actual operation, the distance 1 from the corner (outer canthus) of the eye to the camera lens is known, the distance C from the camera image sensor to the camera lens is also known, the pixel distance A of the palpebral fissure height can be obtained from the first eye position image, and the width and length of a single pixel are also known and are fixed values, so the value of the palpebral fissure height can be calculated.

In the process of training the neural network, the definition of loss function is introduced in order to verify the correctness of the final output or result after training the neural network. The loss function is a function that calculates the distance (e.g., the statistical distance) between the current and expected outputs of the neural network, which provides a numerical indicator of the model's performance. The loss function is an important component of neural networks. A compound loss function may be used in this application, which includes a focal loss, a generalized dice loss, a surface loss and a boundary aware loss.

Firstly, the meaning of the parameters TP, FP, TN and FN in the calculation of loss function should be introduced:

TP: True Positive, which indicates that the predicted result is positive and the actual result is also positive; that is, the prediction is correct;

FP: False Positive, which indicates that the predicted result is positive and the actual result is negative; that is, a positive result is predicted incorrectly and no negative result is predicted;

TN: True Negative, which indicates that the predicted result is negative and the actual result is also negative; that is, the negative result is predicted correctly; and

FN: False Negative, which indicates that the predicted result is negative and the actual result is positive; that is, a negative result is predicted incorrectly, and a positive result is not predicted.

Generalized Dice Loss (GDL) is derived from the F₁ score as specified in Formula (2) below, as a supplement to ordinary accuracy. While common accuracy can be a good indicator of a model's performance, it may not be sufficient when it comes to unbalanced data. The commonly-used loss function in semantic segmentation schemes is the Intersection over Union (IoU) loss, that is, the ratio of intersection over union between one or more “bounding boxes” and one or more “ground truths.” IoU is defined by Formula (1) below. The Dice Factor specified in Formula (3) below can be derived from Formulas (1) and (2), and the Dice Loss in Formula (4) below can be derived from the Dice Factor. Generalized dice loss is just an extension of Dice Loss. Since a simple Dice Loss applies to only two split classes, the generalized dice loss is used to deal with multiple split classes. In the case of this example, the multiple split classes are the five split classes mentioned in the label section discussed above. The formulas are as follows:

$$IoU = \frac{Intersection}{Union} = \frac{TP}{FP + TP + FN} \qquad ( 1 )$$

$${F}_{1} = 2 \times \frac{Precision \times Recall}{Precision + Recall} = \frac{2 TP}{2 TP + FP + FN} \qquad ( 2 )$$

$$Dice Factor = \frac{2 Intersection}{Intersection + Union} \qquad ( 3 )$$

$$Dice Loss = 1 - Dice Factor \qquad ( 4 )$$

wherein Precision refers to the precision of classification model indicators, Recall refers to the recall rate, Intersection refers to the intersection, and Union refers to the union.

Focal Loss is a variation of standard cross-entropy loss in that the focal loss attempts to focus on hard negative samples. The focal loss function can be derived from the following aspects: P_{T} is defined in Formula (5) below, where P stands for a ground truth. With the definition of composite P_{T}, focal loss can be described by Formula (6) below, where αᵣ and γ are hyperparameters. When γ is zero, the focal loss collapses into a cross entropy loss. γ is 2 in this example, and γ provides a moderating effect that takes losses from easy examples when the ground truth is small. As the model learns the easy examples, the function moves to the harder ones. The formulas involved are as follows:

$${P}_{T} = \{ \begin{matrix}P & if y = 1 \\ 1 - P & otherwise\end{matrix} \qquad ( 5 )$$

$$FL ( {T}_{P} ) = - {{a}_{T} ( 1 - {P}_{T} )}^{γ} log ( {P}_{T} ) \qquad ( 6 )$$

The above y specifies the accuracy class (ground truth) label of the training set for the classification of supervised learning techniques, and p is the estimated probability of the label y=1.

A semantically defined boundary in a Boundary Aware Loss (BAL) is a separated area based on a class label. The loss of each pixel is weighted according to its distance from the two nearest fragments, introducing edge awareness. In this application, the Canny edge detector in the open cv is used to generate the boundary pixel, which is further enlarged by two pixels to reduce confusion at the boundary. The value of these boundary pixels may be amplified a certain number of times, and then added to a traditional standard cross-entropy loss entropy to increase the model's attention to the boundary.

Surface Loss (SL) is a statistical distance metric based on the image contour space that preserves small, infrequent structures of high semantic value. BAL tries to maximize the probability of correct pixels near the boundary, while GDL provides a stable gradient for unbalanced conditions. In contrast to BAL and GDL, SL measures the loss of each pixel based on its distance from the ground-truth boundary of each category, effectively restoring small areas that are overlooked by area-based losses. Surface loss calculates the distance of a single pixel to the boundary of each label group and normalizes this distance against the size of the image. These calculated results are combined with the results predicted by the model, and the mean is obtained.

The final compound loss function is given in Formula (7) as follows:

$$Compound loss = {a}_{1} \times Surface loss + {a}_{2} \times Focal loss + {a}_{3} \times Generalized Die Loss + {a}_{4} \times Boundary Aware Loss \times Focal loss \qquad ( 7 )$$

where the variables α₁, α₂, α₃ and α₄ are hyperparameters. In the specific circumstances of this application, α₁ relates to the number of sessions during the training period; α₂ is 1; α₃ is 1−α₁; and α₄ is 20.

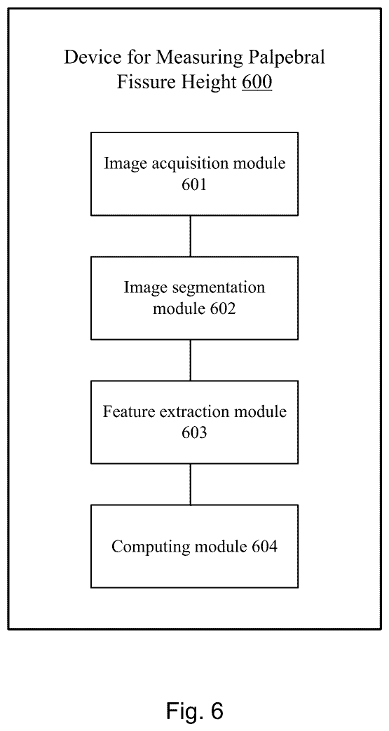

On the one hand, as shown in FIG. 6, this application provides a device 600 for measuring palpebral fissure height, including an image acquisition module 601, an image segmentation module 602, a feature extraction module 603 and a calculation module 604.

The image acquisition module 601 is configured to acquire the first eye position image of the user looking straight ahead in the near-infrared light field of 700-1200 nm in front of the eye.

The image segmentation module 602 adopts the training mode of the neural network to segment the background, the iris, the sclera and the pupil from the first eye position image.

The feature extraction module 603 is configured to extract the pupil center from the partitioned pupil and obtain the pupil center line in the vertical direction.

The computing module 604 is configured to calculate the distance between the junction point of the sclera, the iris or the pupil on the center line of the pupil and the background, and to calculate the palpebral fissure height using this distance.

On the one hand, the present application provides a computer-readable storage medium in which at least one program code is stored, and the at least one program code is loaded and executed by a processor to perform the present method of measuring the palpebral fissure height.

In the exemplary embodiment, a computer readable storage medium is also provided, including a memory storing at least one program code, which is loaded by a processor and executed to perform the method of measuring the palpebral fissure height in the embodiment. For example, the computer readable storage medium can be a Read-Only Memory (ROM), a Random Access Memory (RAM), a Compact Disc Read-Only Memory (CDROM), a magnetic tape, a floppy disk, optical data storage devices, etc.

A person of ordinary skill in the art may understand that all or part of the steps to implement the above embodiments may be performed by hardware, or by a program in hardware related to at least one program code, which may be stored in a computer readable storage medium, such as read-only memory, disk or optical disc.

The above is only a preferred embodiment of this application and is not intended to limit this application. Any modification, equivalent replacement, improvement, etc. made within the spirit and principles of this application shall be covered by this application.

What is claimed is:

1. A method for measuring palpebral fissure height, using a camera to obtain an eye image, comprising:
acquiring a first eye position image of a user in front of an eye, looking straight ahead in a near-infrared light field of 700-1200 nm;
segmenting a background, an iris, a sclera and a pupil from the first eye position image by a training method of a neural network;
extracting a center of the pupil from the segmented pupil and obtaining a center line of the pupil in a vertical direction; and
determining a distance between a junction point of the sclera, the iris or the pupil on the center line of the pupil and the background, and calculating the palpebral fissure height using the distance.

2. The method according to claim 1, wherein the pupil center is an average value of X and Y coordinates of all pixels of the pupil segmented from the first eye position image, and the center line of the pupil is obtained by drawing a vertical line through the average value of the X coordinates.

3. The method according to claim 1, wherein the training method of the neural network segments the background, the iris, the sclera and the pupil from the first eye position image by:
adopting a combination of UNet and DenseNet neural network models as a model for the neural network,
taking the first eye position image as an input to the model for the neural network,
adopting the UNet neural network model to reduce and increase a dimension of the input,
transmitting an output of a dimensionality reducing block to a corresponding dimensionality increasing block by a skip connection, and
each dimensionality decreasing block and upsampling each dimensionality increasing block using the DenseNet neural network model.

4. The method according to claim 3, further comprising verifying a training effect of the neural network by a loss function, wherein the loss function is a compound loss that comprises a focal loss, a generalized dice loss, a surface loss and a boundary aware loss.

5. The method according to claim 4, wherein the compound loss is calculated as follows:
$$compound loss = {a}_{1} \times surface loss + {a}_{2} \times focal loss + {a}_{3} \times generalized dice loss + {a}_{4} \times boundary aware loss \times focal loss$$
wherein α₁, α₂, α₃ and α₄ are hyperparameters, α₁ is related to a number of duration(s) during training, α₂=1, α₃=1−α1, and α₄=20.

6. The method according to claim 1, wherein the palpebral fissure height is:
Palpebral fissure height (B)=a pixel distance (A) of the palpebral fissure height×a single pixel width or length×a distance (D) from the palpebral fissure to a lens of the camera÷a distance (C) from a film in the camera to the lens of the camera,
wherein the pixel distance (A) of the palpebral fissure height is a distance between the junction point of the sclera, the iris, or the pupil on the center line of the pupil and the background.

7. The method according to claim 6, wherein the distance (D) from the palpebral fissure to the lens of the camera is the distance from a corner of the eye to the lens of the camera (1), minus an ocular prominence or exophthalmia (2).

8. The method according to claim 7, wherein the ocular prominence or exophthalmia (2) is an average of a normal eye exophthalmia.

9. A device for measuring palpebral fissure height, comprising:
an image acquisition module, configured to acquire a first eye position image of a user looking straight ahead in a near-infrared light field of 700-1200 nm in front of an eye;
an image segmentation module, configured to adopt a training mode of a neural network to segment the background, the iris, the sclera and the pupil from the first eye position image;
a feature extraction module, configured to extract a pupil center from the segmented pupil and obtain a pupil center line in a vertical direction; and
a computing module, configured to determine a distance between a junction point of the sclera, the iris or the pupil and the background on the pupil center line, and calculate the palpebral fissure height from the distance.

10. A computer-readable storage medium, configured to have at least one program code that is loaded and executed by a processor to perform the method described in claim 1.
